(19) United States
(12) Patent Application Publication
(10) Pub. No.: US 2022/0296392 A1
(43) Pub. Date: Sep. 22, 2022

(54) ARTIFICIAL HAND WITH EXTENDABLE DIGITS

(71) Applicant: ARALLAB MEDIKAL ANONIM SIRKETI, Besiktas (TR)

(72) Inventor: Ilhan Ozan ARAL, Besiktas (TR)

(73) Assignee: ARALLAB MEDIKAL ANONIM SIRKETI, Besiktas (TR)

(21) Appl. No.: 17/839,318

(22) Filed: Jun. 13, 2022

Related U.S. Application Data
(63) Continuation of application No. PCT/TR2020/051263, filed on Dec. 9, 2020.

(30) Foreign Application Priority Data
Dec. 13, 2019 (TR) 2019/20169

(51) Int. Cl. A61F 2/58; A61F 2/72

(52) U.S. Cl. CPC A61F 2/586; A61F 2/72; A61F 2002/5038

(57) ABSTRACT

An artificial hand suitable for use in humans and robots, includes at least one finger member and at least one thumb member, which is actuated by bending the at least one joint by the motor unit; said finger member comprising one proximal and one middle phalanx consisting of two subparts each, said thumb member comprising one proximal and one middle phalanx consisting of two subparts each, and said two-piece proximal and middle phalanges comprise at least one connection means in grooves extending longitudinally therein, increasing lengths of said proximal and middle phalanges by enabling the two pieces to be set apart from each other.

### PRIORITY

This application is a continuation of PCT/TR2020/051263, filed 9 Dec. 2020, which take priority from Turkish Patent Application 2019/20169 filed 13 Dec. 2019, both of which are entirely incorporated by reference.

### TECHNICAL FIELD

The present disclosure generally relates to an artificial hand that comprises thumb and other finger members that may be used as a prosthesis by robots or humans, enabling them to effectively perform daily activities such as gripping and holding actions. The present disclosure relates more specifically to an artificial hand, the size of which can be changed by means of an adjustable thumb and other finger members.

### BACKGROUND

Artificial and prosthetic limbs are used by robots and humans alike as end devices to interact with their environment through surfaces and objects by touching, holding, gripping, and placing. Amputations can occur following disabling injuries to individuals of all ages. Such may particularly happen to an individual at a very young age, or there may be congenital problems resulting in limb loss, calling for many different sizes and proportions of artificial limbs. Since the paradigm of a prosthetic limb in which one size fits all is not practical, the known technique is limited to prostheses of certain sizes. Artificial prostheses are divided into three categories; bionic prostheses that comprise motors, electronics and biosensors, mechanical prostheses that utilize muscle strength and a functional mechanical hand, in which muscle strength is transferred to the mechanical hand, and passive prostheses which are non-functional and are used solely for aesthetical purposes.

The state of the art includes different applications for these three different types of prostheses. One such document; US 2017049583 A1 teaches a hand activated by cables, which can realize different types of grip motions by moving each finger, which are moved by tendon lines connected by rods. A thumb has its own orientation cable, which allows it to touch the tip of the index finger. The cable, energized from the body, performs inter-finger position variations for powerful and accurate grips.

The prosthetic hand disclosed in document WO 2017111582 A1 is another teaching known in the art. It is characterized by the finger elements consisting of a stick attached to a palm and joints as a structure supported by a wrist. Fingers can take multiple loaded positions depending on the size of the load to be applied on the inside of the hand; and in the opposite situation, the finger elements close towards the hand and a locking mechanism locks the structure into one of the multiple loaded positions.

U.S. Pat. No. 9,333,096 B2, yet another document known in the art, discloses a method of increasing the reach of the limb by including multiple extensible segments of a prosthetic limb. In one aspect of the invention, one of these extendable segments is constructed to be analog with the humerus bone of the human arm, and the other with the radius and ulna bones of the human arm. In another aspect of the invention, an end assembly, which is connected to the equivalent of the extensible segments in question, can also be extended transversely to extend the diameter of the limb.

According to JP 2012187426 A, a hand with two different joint locations is proposed. These two joints can be expressed as a metacarpophalangeal joint and an interphalangeal joint connected by a link mechanism; The bending and opening movements of these joints are designed to be simultaneous.

### SUMMARY

A feature of the prothesis disclosed herein is to provide a scalable artificial hand with extendable fingers that can be used by both robots and humans.

Another feature of the prothesis disclosed herein is to provide an artificial hand which can be enlarged entirely and proportionally, usable by both robots and humans.

A further feature of the prothesis disclosed herein is to provide an artificial hand with extendable fingers and 3D printable outer parts, usable by both robots and humans.

A still further feature of the prothesis disclosed herein is to provide a prosthetic arm with extendable fingers and characterized by the adjustability of digits to desired dimensions suitable for the use of people who have undergone transradial or transhumeral amputations.

The prosthesis disclosed is an artificial hand suitable for cases of upper extremity limb loss. Said prosthesis comprises extendable parts, thus is capable of offering an adjustable size. For a prosthetic upper-body limb in the present invention, the artificial hand may be used as a prosthetic hand with finger elements containing multiple different sub modules analogously matching the finger bones in the real human hand.

Contrary to existing artificial/prosthetic hand or upper limb prostheses known in the art, which are primarily designed for people who have stopped skeletal growth or ones more suitable for temporary use, the artificial hand for the upper extremity limbs disclosed herein addresses the need for suitable prosthesis forms that match the physiological proportions of an adult and/or a child. The prothesis disclosed herein also aims to address young people with upper extremity prosthetics who experience physical asymmetry and functional impairment of used prosthesis resulting from the fact that they almost exclusively outgrow their first prosthesis with age.

Finger elements disclosed as part of the prothesis disclosed herein comprise proximal, middle and distal phalanx submodules, among which the proximal and middle phalanx submodules further comprise at least one extension rod that allows them to extend, thus enlarge the fingers and, consequently, the hand. As a direct result of this innovative aspect, the finger joints could be moved away from each other as the phalanges are extended, allowing the user to have a larger hand that can be used for an extended period of time, eliminating the need for costly renewal, replacement or adaptation.

The prothesis disclosed herein also aims to overcome the weight and cost problem found in many prostheses known in the art. The solution put forth, through the choice of material and designs; is maintaining the functionality of a prosthesis capable of retaining harmony with the user's body even if the skeletal growth proceeds or comes to a halt. Thus, even if the need of a prosthesis emerges at an early age, disclosed prosthesis may also be used in the later stages of physical development, in addition to providing prostheses of reasonable weight for individuals in their childhood.

### BRIEF DESCRIPTION OF THE FIGURES

Accompanying drawings are given solely for the purpose of exemplifying an artificial hand with extendable fingers, whose advantages over prior art were outlined above and will be explained in brief hereinafter.

The drawings are not meant to delimit the scope of protection as identified in the claims nor should they be referred to alone in an effort to interpret the scope identified in said claims without recourse to the technical disclosure in the description herein.

FIG. 1 demonstrates the top perspective view of an example proposed artificial hand, with 3D-printed cover parts on, according to an embodiment.

FIG. 2 demonstrates a bottom view of the proposed artificial hand, with its 3D-printed cover parts on, according to an embodiment.

FIG. 3 demonstrates a top perspective view of the skeletal mechanism of the proposed artificial hand, according to an embodiment.

FIG. 4 demonstrates an exploded top perspective view of the skeletal mechanism of the proposed artificial hand, according to an embodiment.

FIG. 5 demonstrates a side view of a finger member skeleton of the proposed artificial hand, according to an embodiment.

FIG. 6 demonstrates top perspective view of a finger member skeleton of the proposed artificial hand, according to an embodiment.

FIG. 7 demonstrates a bottom perspective view of a finger member skeleton of the proposed artificial hand, according to an embodiment.

FIG. 8 demonstrates top perspective view of a finger member skeleton of the proposed artificial hand in the bent/squeezed position, according to an embodiment.

FIG. 9 demonstrates top perspective view of a finger member skeleton of the artificial hand in its extended state, according to an embodiment.

FIG. 10 demonstrates top perspective view of the extended and original states of the finger member skeletons of the proposed artificial hand according to an embodiment.

FIG. 11 demonstrates top perspective view of the extended and original states of finger members with covers on, according to an embodiment.

FIG. 12 demonstrates top perspective view of a thumb member of the proposed artificial hand, according to an embodiment.

FIG. 13 demonstrates an alternative top perspective view of a thumb member of the proposed artificial hand, according to an embodiment.

FIG. 14 demonstrates top perspective view of a thumb member of the artificial hand, with flexion mechanism thereof exploded, according to an embodiment.

FIG. 15 demonstrates top perspective view of a thumb member of the proposed artificial hand in adduction position, according to an embodiment.

FIG. 16 demonstrates top perspective view of a thumb member of the proposed artificial hand in abduction position, according to an embodiment.

FIG. 17 demonstrates top perspective view of an unbent thumb member of the proposed artificial hand with covers on, according to an embodiment.

FIG. 18 demonstrates exploded top perspective view of an unbent thumb member skeleton of the proposed artificial hand, according to an embodiment.

FIG. 19 demonstrates exploded top perspective view of a thumb member skeleton of the proposed artificial hand, according to an embodiment.

FIG. 20 demonstrates top perspective view of a bent thumb member skeleton of the proposed artificial hand, according to an embodiment.

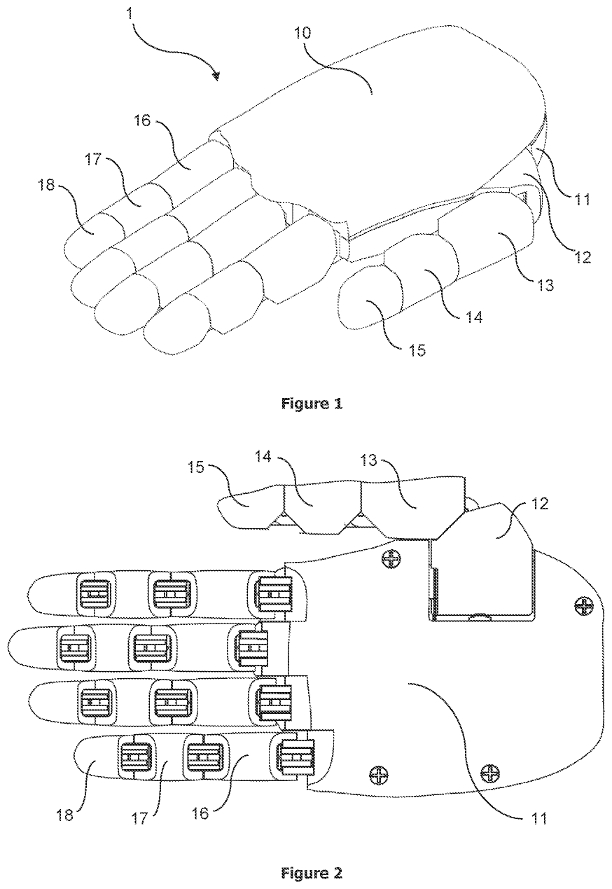

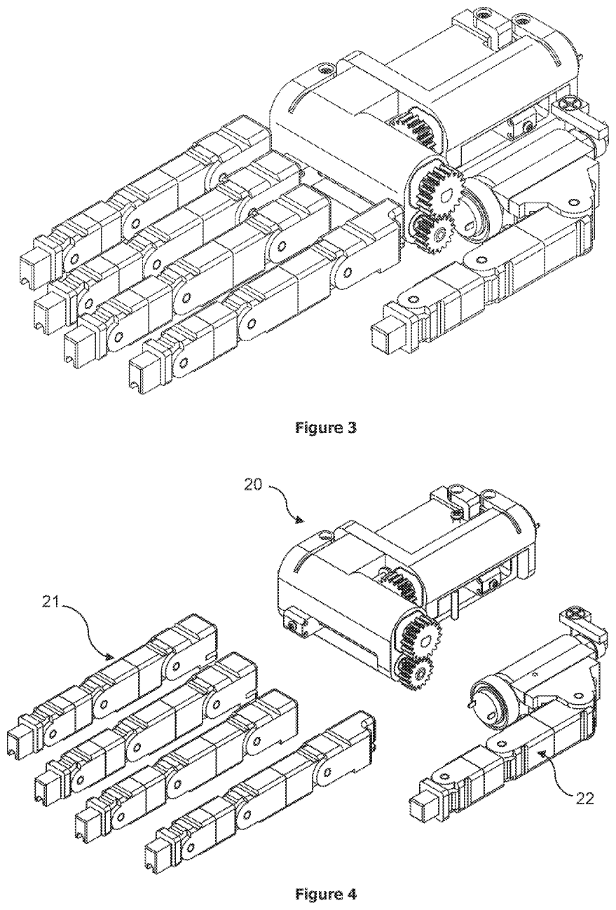

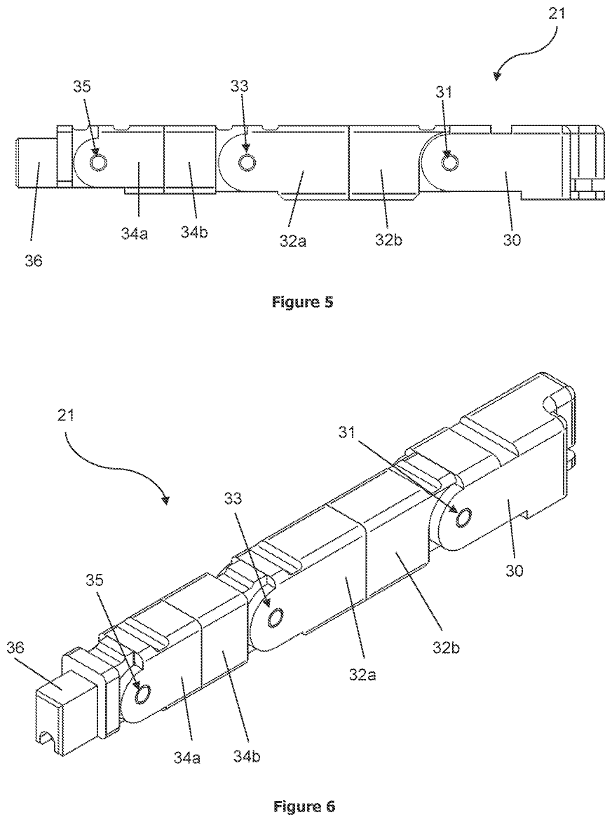

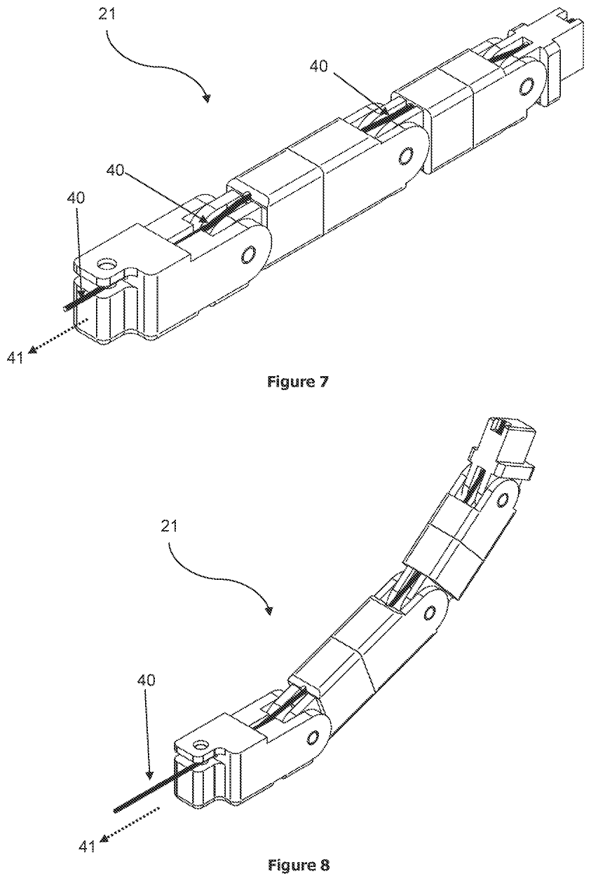

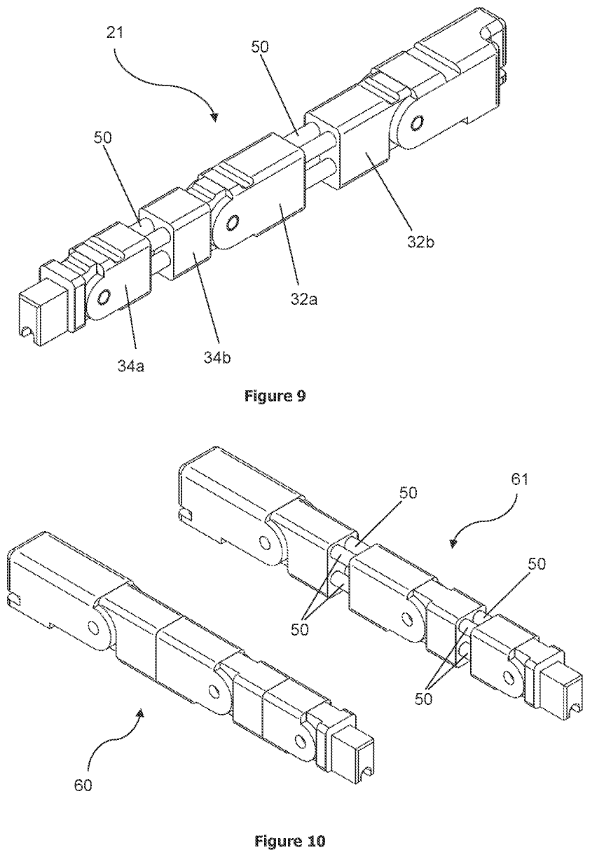

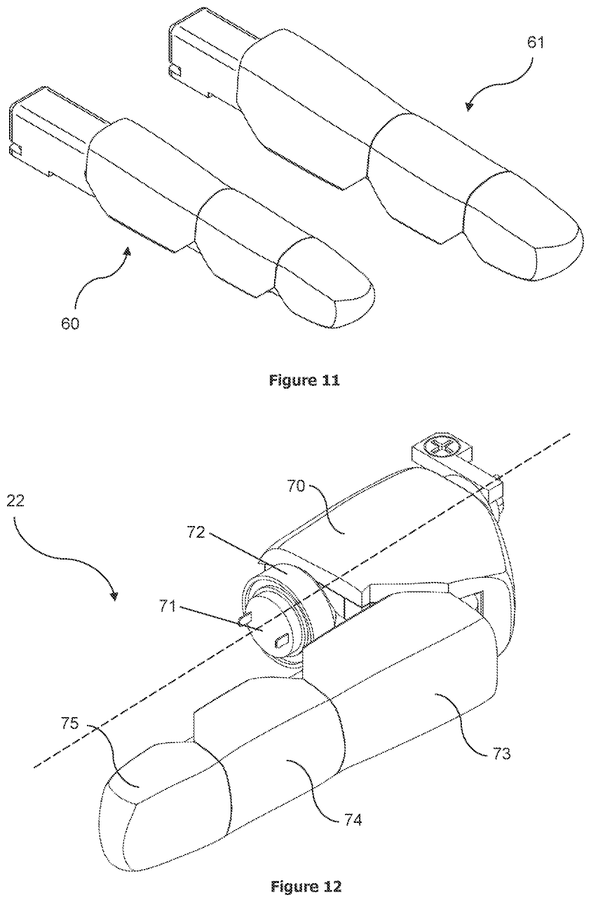

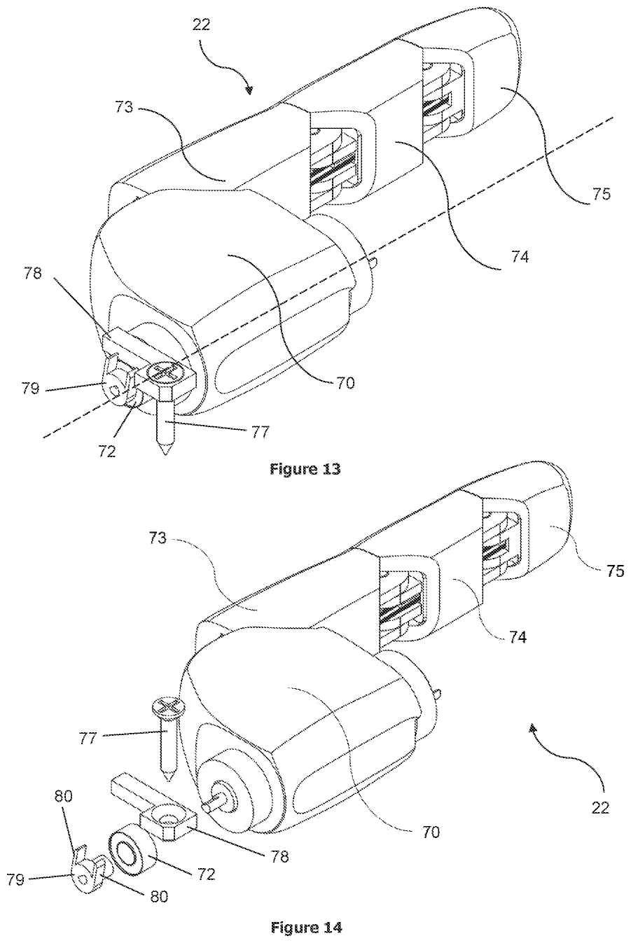

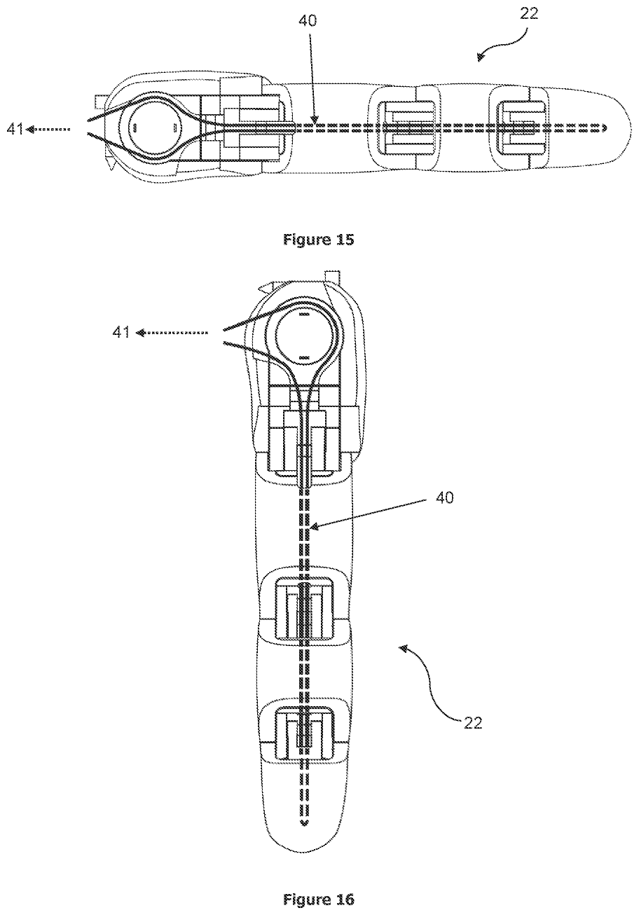

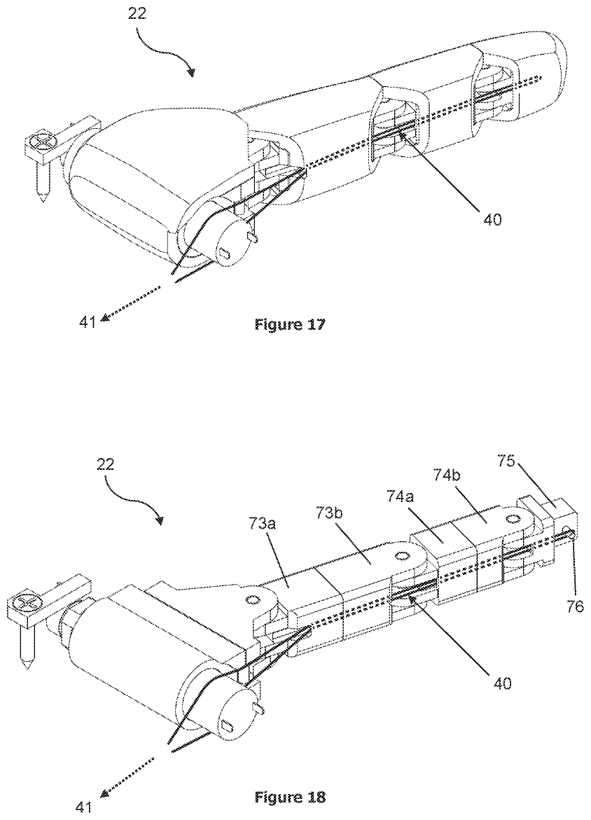

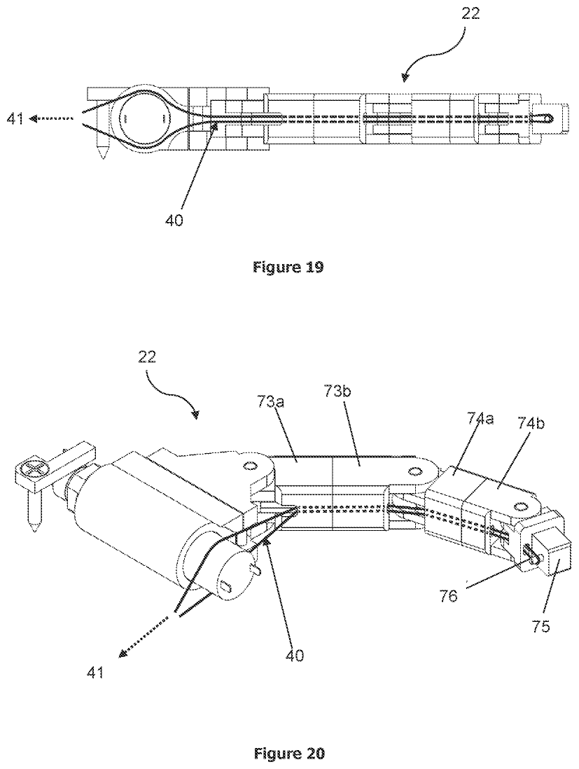

### DETAILED DESCRIPTION

The following numerals are referred to in the detailed description:
1) Artificial hand
10) Palm cover top
11) Palm cover bottom
12) Thumb trapezium
13) Thumb proximal cover
14) Thumb middle cover
15) Thumb distal cover
16) Finger proximal cover
17) Finger middle cover
18) Finger distal cover
20) Motor unit
21) Finger member
22) Thumb member
30) Palm joint
31) Proximal joint pin
32ab) Proximal phalanx
33) Middle joint pin
34ab) Middle phalanx
35) Distal joint pin
36) Distal phalanx
40) Bending means
41) Pull direction
50) Connection means
60) Standard finger member
61) Extended finger member
70) Thumb metacarpal
71) Motor
72) Bearing
73ab) Proximal Phalanx
74ab) Middle Phalanx
75) Distal Phalanx
76) Hook
77) Key stabilizing screw
78) Key stabilizer
79) Flexible key
80) Flaps

An artificial hand (1) for a prosthetic upper body limb, as disclosed, comprises extendable parts which offers the benefit of offering an adjustable size. When the artificial hand in the prosthetic upper body limb of the present invention is examined in more detail, it reveals an artificial hand (1) with finger members comprising multiple different sub-modules that correspond analogously to the finger bones in the real human hand.

In contrast to the existing artificial/prosthetic hand or upper limb prosthetic solutions in the art which are mainly designed for people who moved past the ages of skeletal growth, the artificial hand (1) for prosthetic upper body limb proposed in the present invention provides solutions to the problem of such known prostheses having a fixed, unalterable form (for either an adult or a child) arising from the manufacturing and design properties thereof. The present invention also aims to address problems of physical asymmetry and functionality loss that are encountered by young people with upper extremity prostheses, since they outgrow their initial prostheses as they age.

According to an embodiment of the artificial hand (1) for upper body limb prosthesis proposed in the present invention, all cover parts may be manufactured using three-dimensional printing technology. Cap and cover parts manufacturable using said three-dimensional printing technology cover the outer part of the artificial hand (1) in a state ready to use. Said covers individually cover skeletal parts of said artificial hand (1), which are according to different embodiments manufactured out of various other material. Said material said skeletal pieces are manufactured therefrom may be suitable metals according to different embodiments of the invention. Said cover parts may be attached to and removed from said skeletal parts, thus bringing replaceability. When said cover parts, finger and thumb members are extended (i.e. lengthwise), they may be adapted to this new dimension and replacement is performed. Thus, all said parts may continue to work harmoniously with one another whilst the core functionality of the artificial hand (1) is fully preserved.

According to an embodiment, the part of the upper body prosthesis that is analogous to the human palm is comprised by a combination formed by a palm cap top (10) and a palm cap bottom (11). According to an embodiment of the invention, said artificial hand (1) may comprise multiple finger members (21) and at least one thumb member (22). According to an embodiment, the artificial hand (1) comprises four finger members (21) and a thumb member (22), substantially similar to the hand of a human person. The parts forming the skeletons of said finger members (21), which will be referred to later, are covered with a finger proximal cover (16), finger middle cover (17) and finger distal cover (18), respectively, from the palm to the fingertip. The parts forming the skeleton of said thumb member (22) are likewise covered with a thumb proximal cover (13), a thumb middle cover (14) and a thumb distal cover (15).

An artificial hand (1) for the prosthesis upper body limb proposed in the present invention comprises at least one motor unit (20) for the task of actuating said multiple finger members (21) and at least one thumb member (22). Said motor unit (20) carries out the actuation tasks for all of the finger and thumb members (21, 22) via tightening and loosening a bending means (40) extending from said finger and thumb members (21, 22). Said bending means (40) is subjected to linear movement according to an embodiment. Thus, said finger and thumb members (21, 22) are rendered open and closed for grip motions.

According to an embodiment, said finger member (21) comprises structural sub-modules that are analogically designed and constructed with respect to bones in a real human finger. In a preferred embodiment, two of said sub-modules are extendable, and are located in the parts corresponding to the proximal and middle phalanges of the real human finger. Said at least one finger member (21) comprises a palm joint (30) that establishes connection thereof with the palm part. Said palm joint (30) is structurally directly related to a proximal phalanx (32) by means of a proximal joint pin (31) whereby the ability to maintain joint rotation is provided. Said proximal phalanx (32) consists of two parts (32a, 32b), which may be set apart from each other by a multiplicity of connection means (50) while preserving its structural integrity. The proximal phalanx (32) is connected to a middle phalanx (34) by means of a middle joint pin (33), which provides another articular rotational ability. Said middle phalanx (34) consists of two parts (34a, 34b), which can be removed from each other by connection means (50) while preserving its structural integrity. The middle phalanx (34) is connected to a distal phalanx (36) by means of a distal joint pin (34), whereby another articular rotation point is provided.

According to an embodiment, said finger member (21) comprises a channel such as a groove facing the inner surface of the hand, so as to accommodate a bending means (40). Said bending means (40) is positioned along said channel, one end of which is connected to a tip of the distal phalanx (36) of said finger member (21) and at the other and to a linear actuator located in the motor unit (20). Exerting motion on said bending means (40) by the linear actuator along the pull direction (41) makes said finger member (21) open and close via the joint function at the vicinity of proximal, middle and distal joint pins (31, 33, 35).

According to an embodiment, with respect to the parts making up the palm section, namely palm cap top (10) and palm cap bottom (11); during the closing movement of the finger member (21), the palm joint (30) remains fixed, the proximal phalanx (32), the middle phalanx (34) and the distal phalanx (36) begin to close inward along the axes of proximal, middle and distal joint pins (31, 33, 35) respectively. The opening movement is realized when these movements occur in the opposite direction following the release of the bending means (40). According to an embodiment of the present invention, there are rotational elements around the proximal, middle and distal joint pins (31, 33, 35), and these elements are forced to open outwards around the axes of said proximal, middle and distal joint pins (31, 33, 35) of the finger element (21). According to an embodiment of the invention, these rotational elements are springs.

According to an embodiment, the proximal phalanx (32) and middle phalanx (34) within the finger member (21) each comprise two sub-parts (32ab) and (34ab) respectively. Said two parts, which are the parts 32a and 32b on the proximal phalanx (32) and the parts 34a and 34b on the middle phalanx (34), are defined by at least one connection means (50) that are situated in grooves extending along their length. Said connecting means (50) may be more than one. According to a preferred embodiment of the invention, said proximal phalanx (32) comprises four connection means (50) between its parts (32ab), and said middle phalanx (34) comprises four connection means (50) between its parts (34ab); thereby making the total number of connection means (50) comprised by one finger member (21) eight. In accordance with an embodiment of the present invention, when finger members (21) are to be extended, said at least one connection means (50) are replaced by another connection means (50) of appropriate length, thereby and increasing the distance between (32ab) and (34ab) and in turn the distance(s) between proximal, middle and distal joint pins (31, 33, 35). According to an embodiment of the present invention, said connecting means (50) is a rod.

According to an embodiment, when parts of said proximal phalanx (32ab) are pulled apart from each other, said at least one connection means (50) comprised by said proximal phalanx (32) may be replaced with a longer structural equivalent, making the proximal phalanx (32) extend in size. In this extended state, said parts of said proximal phalanx (32a, 32b) and said at least one connection means (50) retain structural integrity such that the form of proximal phalanx (32) is retained. In other words, said at least one connection means (50) may be selected and utilized according to a desired size insofar as it keeps physical functions of the proximal phalanx fragments (32a, 32b) while making it longer.

According to an embodiment, when parts of said middle phalanx (34ab) are pulled apart from each other, said at least one connection means (50) comprised by said middle phalanx (34) may be replaced with a longer structural equivalent, making the middle phalanx (34) extend in size. In this extended state, said parts of said middle phalanx (34a, 34b) and said at least one connection means (50) retain structural integrity such that the form of middle phalanx (34) is retained. In other words, said at least one connection means (50) may be selected and utilized according to a desired size insofar as it keeps physical functions of the middle phalanx fragments (32a, 32b) while making it longer.

According to an embodiment, after the finger member (21) is extended, finger proximal cover (16) and finger middle cover (17) parts prepared according to the new phalanx lengths are attached to proper positions thereof on the artificial hand (1), ready for use.

According to an embodiment, said thumb member (22) comprises structural sub-units that are designed analogously to thumb bones of a human hand. Two of these sub-units are extendable and are located in the parts corresponding to the proximal and middle phalanges of the real human thumb. The at least one thumb member (22) comprises a thumb metacarpal (70) that connects said at least one thumb member (22) to the palm section of said artificial hand (1). Said thumb metacarpal (70) is equipped with two bearings (72) allowing said thumb metacarpal (70) to be connected with the palm top and bottom covers (10, 11), respectively. Inside said bearing (72) that is facing the back of the wrist, there is a motor (71) to enable adduction and abduction movements to be performed by said thumb member (22). Said thumb metacarpal (70) is connected to a proximal phalanx (73) via a proximal joint pin, whereby the ability to develop joint rotation is provided. The said proximal phalanx (73) comprises two parts (73a, 73b), which may be moved apart from each other by replacement of said connection means (50) contained therebetween in a fashion principally identical to the case with finger members, while preserving structural integrity. Said proximal phalanx (73) is connected to a middle phalanx (74) by means of a middle joint pin, where another joint with rotation ability is formed. Said middle phalanx (74) comprises two parts (74a, 74b) which may be moved apart from each other by replacement of said connection means (50) contained therebetween in a fashion principally identical to the case with finger members, while preserving structural integrity. The middle phalanx (74) is connected to a distal phalanx (75) by means of a distal joint pin, whereby another articular rotation ability is provided.

According to an embodiment, said thumb member (22) comprises a channel which is located in the part facing the inside of the hand to accommodate a bending means (40), such as a wire. Said bending means (40) is positioned through said channel in a way such that it allows said bending means (40) to be wrapped around a hook (76) at the end of the distal phalanx (75) part of said thumb member (22) and brought back to the base; while one of the two ends of said bending means (40) is configured to pass underneath said motor (71) and the other end is configured to pass over the motor unit (20). Moving said bending means (40) along the pull direction (41) by said motor unit (20) allows said thumb member (22) to be closed and opened, which is facilitated by means of the joint function in the positions of the proximal, middle and distal joint pins.

According to an embodiment, transmitting the bending means (40) in said thumb member (22) to the motor unit (20) passing through the top and bottom thereof in both of the adduction or abduction movements provides the benefit of said bending means (40) not experiencing elongation or shortening. In other words, excessive tightening and loosening in the adduction and abduction movements of said bending means (40) that would happen in the presence of a single-line bending means (40), i.e., one that is not wrapped around the hook (76) and back (as is the case with finger member(s) (21)) is prevented.

According to an embodiment, in the event that said thumb member (22) is subject to an overly large amount of torque (e.g. an impact) or load during movement around the axis of rotation, said flaps (80) on said flexible key (79) are configured to be able to stretch so as to absorb a sufficient amount of this force. Thus, the shape of the artificial hand (1) and the gears in the motor (71) are protected against any possible structural damage from this excessive torque.

According to an embodiment, an artificial hand (1) suitable for use in humans and robots, comprising at least one finger member (21) actuatable via bending of at least one joint (40) and at least one thumb member (22) actuatable via bending of at least one joint (40) is proposed.

According to another embodiment, said at least one finger member (21) further comprises one proximal and one middle phalanx (32, 34) parts, each comprising two subparts.

According to another embodiment, said at least one thumb member (22) further comprises one proximal and one middle phalanx (73, 74) parts, each comprising two subparts.

According to yet another embodiment, said two-subpart proximal phalanx (32, 34) and middle phalanx (73, 74) members comprise at least one connection means (50) housed in longitudinally extending grooves therein, whereby said proximal and middle phalanges (32, 34; 73, 74) may be elongated via replacement of said connection means (50) with longer equivalents, setting apart said subparts of said proximal and middle phalanges whilst maintaining structural integrity.

According to another embodiment, said artificial hand (1) comprises a motor unit (20) for actuating said at least one finger member (21) and at least one thumb member (22).

According to another embodiment, each of said proximal and middle phalanges (32, 34, 73, 74) comprise four connecting means (50).

According to another embodiment, said at least one finger member (21) and said at least one thumb member (22) further comprise a bending means (40) facilitating opening and closing thereof via being actuated by said motor unit (20) along a pull direction (41).

According to another embodiment, said at least one thumb member (22) further comprises a motor (71) on a thumb metacarpal (70), whereby adduction and abduction motions are effectuated.

According to another embodiment, said at least one thumb member (22) further comprises a flexible key (79) characterized by multiple flaps (80) whereby damage due to excessive torque is prevented.

According to another embodiment, said at least one thumb member (22) further comprises a hook (76) situated on the tip of a distal phalanx (75) element whereby excessive stretching and loosening of said bending means (40) is prevented.

1. An artificial hand suitable for use in humans and robots, comprising: at least one finger member actuatable via bending of at least one joint and at least one thumb member actuatable via bending of at least one joint wherein:
said at least one finger member comprising one proximal phalanx part and one middle phalanx part, each comprising two subparts,
said at least one thumb member comprising one proximal phalanx part and one middle phalanx part, each comprising two subparts, and;
said proximal phalanx part and said middle phalanx part each comprise at least one connection means, and each of said two subparts comprise longitudinally extending grooves in which said at least one connection means is housed to maintain respective structural integrity between the two subparts of each of said proximal phalanx part and said middle phalanx part, wherein said proximal phalanx part and said middle phalanx part are elongatable via replacement of said connection means with longer equivalents, setting apart said two subparts of said proximal phalanx part and said middle phalanx part whilst maintaining the respective structural integrity.

2. The artificial hand as set forth in claim 1, further comprising a motor, said at least one finger member and said at least one thumb member actuatable by said motor.

3. The artificial hand as set forth in claim 1, wherein each of said proximal phalanx part and said middle phalanx part comprise four connecting means.

4. The artificial hand as set forth in claim 2, wherein said at least one finger member and said at least one thumb member further comprise a bending means, the bending means actuatable with said motor along a pull direction to facilitate opening and closing of said at least one finger member and said at least one thumb member.

5. The artificial hand as set forth in claim 4, wherein said bending means comprises a wire and said pull direction represents linear movement of said wire.

6. The artificial hand as set forth in claim 5, wherein said wire is tightened and loosened by said motor along said pull direction to facilitate respective closing and opening of said at least one finger member and said at least one thumb member.

7. The artificial hand as set forth in claim 5, wherein said at least one finger member and said at least one thumb member each comprises a hook at a distal end of said at least one finger member or said at least one thumb member, and said wire is wrapped around said hook to provide two lines of wire extending linearly along said at least one finger member and said at least one thumb member.

8. The artificial hand as set forth in claim 1, wherein said at least one thumb member further comprises a motor and a thumb metacarpal, the motor and the thumb metacarpal cooperatively operable to effectuate adduction and abduction motions of said thumb member.

9. The artificial hand as set forth in claim 1, wherein said at least one thumb member further comprises a flexible key comprising multiple flaps that stretch to absorb force and prevent damage due to excessive torque on said at least one thumb member.

10. The artificial hand as set forth in claim 4, wherein said at least one thumb member further comprises a distal phalanx part and a hook situated on a tip of the distal phalanx part to receive said bending means such that excessive stretching and loosening of said bending means is prevented.

11. The artificial hand as set forth in claim 1, wherein said connection means is an extension rod having a first end and a second end, and said longitudinally extending grooves include respective apertures in each of said two subparts, the respective apertures being sized and alignable to respectively receive said first end and said second end.
